(19) United States
(12) Patent Application Publication
(10) Pub. No.: US 2003/0093007 A1
(43) Pub. Date: May 15, 2003

(54) Biopsy apparatus with radio frequency cauterization and methods for its use

(75) Inventor: Bradford J. Wood, Potomac, MD (US)

Correspondence Address: KLARQUIST SPARKMAN, LLP, One World Trade Center, Suite 1600, 121 S.W. Salmon Street, Portland, OR, 97204, US

(73) Assignee: The Government of the U.S.A., as represented by the Secretary, Department of Health and Human Serv

(21) Appl. No.: 10/274,074

(22) Filed: Oct. 17, 2002

Related U.S. Application Data
(60) Provisional application No. 60/330,298, filed on Oct. 17, 2001.
(60) Provisional application No. 60/370,965, filed on Apr. 8, 2002.

(51) Int. Cl. A61B 10/00

(52) U.S. Cl. 600/564

(57) ABSTRACT

A needle biopsy system, utilizing a removable inner stylet and an outer cannula sized for receiving the stylet, capable of cauterizing a biopsy track is disclosed. The cannula is capable of conducting radio frequency or other cauterizing energy. A portion of the outer surface of the cannula is insulated, allowing the exposed portion to contact adjacent tissue when inserted into the body of a subject. The apparatus can be used to perform a percutaneous biopsy of an organ or tissue. After the biopsied tissue is removed, the apparatus is pulled back through the biopsy track and cauterization energy is applied to the exposed cannula portion causing cauterization and coagulation of the tissue adjacent to the needle track.

### CROSS-REFERENCE TO RELATED APPLICATIONS

This application claims the benefit of both U.S. Provisional Patent Application No. 60/330,298, filed Oct. 17, 2001, herein incorporated by reference in its entirety, and U.S. Provisional Patent Application No. 60/370,965, filed Apr. 8, 2002, herein incorporated by reference in its entirety.

### FIELD

This invention relates to percutaneous surgical devices, such as devices for performing percutaneous biopsies.

### BACKGROUND

Percutaneous image-guided needle biopsy is one of the most common invasive procedures performed by many radiologists including body imagers, vascular and interventional radiologists, ultrasonographers, and general diagnostic radiologists. This procedure is often performed in highly vascularized organs or in tumors with rich macroscopic and microscopic blood supply, often due to tumor angiogenesis. Bleeding complications present one of the more common risks to the patient undergoing a needle biopsy or aspiration. Hemorrhage following biopsy often goes unnoticed unless the patient develops alterations in hemodynamic status due to the blood loss. If the operator notices a large amount of blood emitting from a biopsy needle, or if a particular tumor is deemed to be at high-risk for hemorrhage, previously, there have been few options to the operator.

The clinical problem of bleeding after percutaneous biopsies is not uncommon. In addition, patients with uncorrectable coagulopathies or those on anticoagulation regimine have higher risk for bleeding during biopsies. The surgeon performing biopsy during open surgery has the option of cauterizing with a cauterization device which visibly stops bleeding under direct visualization. However, the vast majority of solid organ biopsies are performed under imaging guidance, usually ultrasound, with no direct visualization option. Thus, the operator not only has no option for cauterizing, but also cannot see whether there is bleeding from the surface of the biopsy region until it has accumulated in significant quantity to become visible on ultrasound. Furthermore, post-biopsy imaging is not routinely performed unless there is hemodynamic compromise or symptoms or other secondary evidence of ongoing bleeding. The percutaneous biopsy can be imaging-guided, but this remains a “blind” procedure with sub-optimal options for controlling bleeding, which usually is inapparent early in the post-biopsy course.

Prior treatment options for bleeding during biopsy included the trans-needle injection of an autologous blood clot or gelfoam collagen pledgets in an attempt to promote clotting and hemostasis in the needle track. Such procedures are performed without uniformity or standard technique and their effectiveness has not been clearly shown, especially in a patient with abnormal clotting or bleeding diathesis or coagulopathy. Furthermore, the use of gelfoam for this indication represents off-label use. The development of an effective, inexpensive and technically simple method of cauterizing a coaxial needle biopsy track would present a solution to this difficult clinical problem.

### SUMMARY

Disclosed is a needle biopsy system capable of cauterizing the needle track after core biopsy in order to reduce the risks of hemorrhage and needle track seeding by tumor cells. In some embodiments, radio frequency (RF) energy is utilized to perform the cauterization. Cauterizing the needle track reduces hemorrhage that occurs at the organ surface as compared to traditional needle biopsy.

Illustrated is a coaxial biopsy system having a removable inner stylet and a cannula sized for receiving the stylet. The cannula is capable of conducting RF energy, and a portion of the outer surface of the cannula is insulated to contain RF energy conducted by the cannula. In some embodiments, the majority of the cannula distal end portion thereof is insulated With the distal end exposed and capable of contacting the surrounding tissue when inserted into the body of a subject. In alternative embodiments, a portion of the cannula outer surface adjacent to the distal end of the cannula is exposed. Any non-conducting material can be used as insulation, such as a non-conducting polymer that insulates the cannula shaft and the tissue immediately in contact therewith.

In some embodiments, the device is used to perform a percutaneous biopsy of an organ or tissue. After the biopsied tissue is removed, the needle is pulled back through the biopsy track and cauterization energy is applied to the exposed cannula tip causing cauterization and coagulation of the tissue adjacent to the needle track. Tissue that is remote from the needle track remains uninjured.

### BRIEF DESCRIPTION OF THE DRAWINGS

FIG. 1 is a side elevation view of one embodiment of the device.

FIG. 2 is an enlarged cross-sectional view taken along line 2-2 in FIG. 1.

FIG. 3A is an enlarged longitudinal cut-away view of the distal end of the device shown in FIG. 1. FIGS. 3B-3G are similar, enlarged longitudinal cut-away views of alternative embodiments of the device.

FIGS. 4A-C are illustrations of one method of taking a tissue biopsy from a subject using the device illustrated in FIGS. 1 and 3A.

### DETAILED DESCRIPTION

The singular forms “a,” “an,” and “the” refer to one or more than one, unless the context clearly indicates otherwise. For example, the term “comprising a needle” includes single or plural needles and is considered equivalent to the phrase “comprising at least one needle.”

The term “or” refers to a single element of stated alternative elements or a combination of two or more elements. For example, the phrase “radio frequency or microwave energy” refers to radio frequency energy, microwave energy, or both radio frequency and microwave energies.

The term “comprises” means “includes.” Thus, “comprising a cannula and a stylet” means “including a cannula and a stylet,” without excluding additional elements.

The term “proximal” refers to a portion of an instrument closer to an operator, while “distal” refers to a portion of the instrument farther away from the operator.

The term “subject” refers to both human and other animal subjects. In certain embodiments, the subject is a human or other mammal such as a primate, cat, dog, cow, horse, rodent, sheep, goat, or pig.

This detailed description discloses a coaxial biopsy apparatus for cauterizing the biopsy track upon withdrawal of the apparatus from the body of the subject. Using the apparatus and method described herein, percutaneous biopsies can coagulate the biopsy track in a reliable, predictable, easy, and inexpensive manner.

The illustrated apparatus is composed of a partially insulated hollow outer needle or cannula, and an inner needle or stylet capable of being inserted and removed from the lumen of the outer needle/cannula. The outer and inner needles can be coaxial, and a portion at, or adjacent to, the distal end of the cannula is left exposed and non-insulated. This exposed portion contacts or comes into proximity with surrounding tissue and, when cauterization energy is supplied to the apparatus, cauterizes or coagulates the surrounding tissue adjacent the apparatus. Thus, this exposed portion functions as a cauterization electrode. This cauterization electrode can be a monopolar electrode (as illustrated), or can be formed into a bipolar electrode by encasing it with a sheath of electrically condutive material, such as metal.

The term “coaxial” describes an apparatus similar to a coaxial cable (which consists of a conductor surrounded by an insulating layer), but is not limited to only an apparatus where the partially insulated hollow outer needle and the inner needle have perfectly coincident longitudinal axes. The longitudinal axes of the inner and outer needles can be offset from one another, even substantially offset, yet still be considered “coaxial,” so long as the inner needle extends slidably through the outer needle and the axes of the inner and outer needles are substantially parallel.

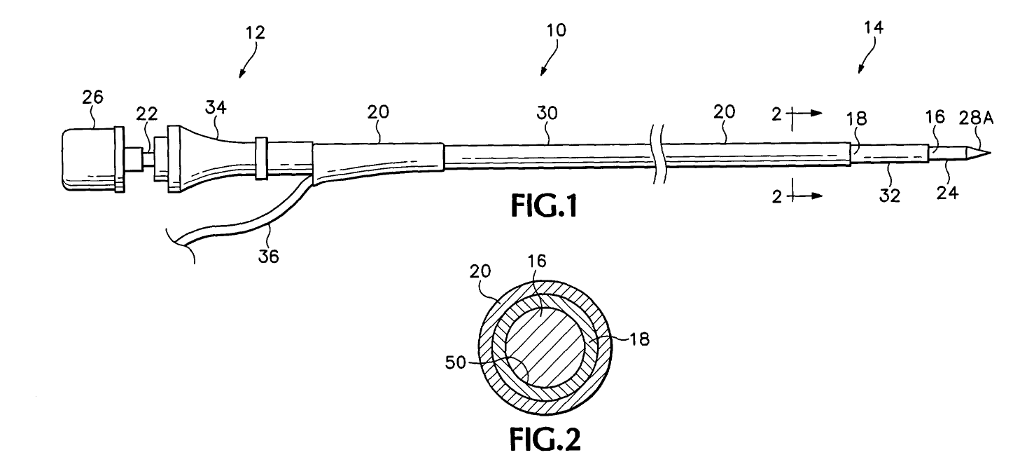

FIG. 1 illustrates an external view of the apparatus 10 having a proximal end 12 and a distal end 14. An elongated needle or stylet 16 is shown extending slidably through cannula 18, which is surrounded over a major portion of its length by an electrically and/or thermally insulating layer 20. This arrangement is also shown in cross-sectional view in FIG. 2. While the illustrated embodiment of the apparatus 10 has a circular cross-section (as particularly illustrated in FIG. 2), alternative embodiments could employ an apparatus having a differently shaped cross-section, such as square, oval, rectangular, or triangular cross-section.

Stylet 16 has a proximal end portion 22 and a distal end portion 24, and can be solid or hollow. In this illustrated embodiment, a grip 26 has been mounted on the proximal end 22 of stylet 16, while distal end 24 of stylet 16 has been formed to a sharpened point 28A. Stylet 16 can be made from any material suitable for percutaneous insertion into the body of a subject, including (but not limited to) surgical steel, a polymer, or composite materials. Stylet 16 can be electrically conductive or nonconductive and can be made from a stiff or flexible material, depending on the needs of the operator.

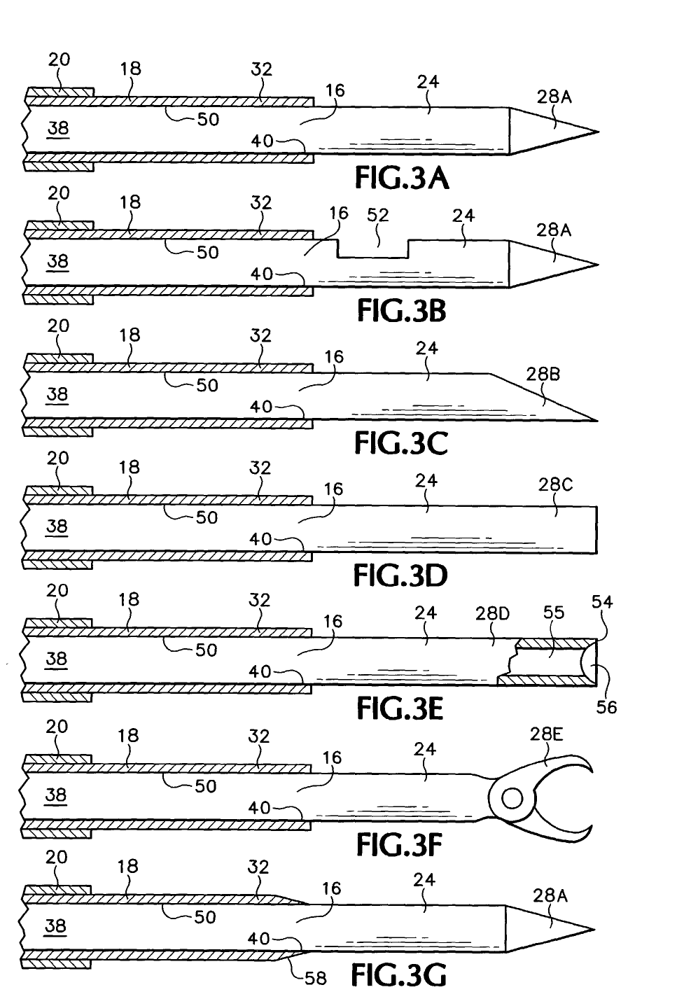

Grip 26 provides a handhold for the operator and assists in the movement of stylet 16, for example, by making it easier for the operator to insert stylet 16 into (or remove it from) cannula 18. Sharpened point 28A provides a simple mechanism for insertion of the device into and through tissue and for biopsying tissue, though alternate embodiments employ other mechanisms for biopsying tissue and other surgical applications, such as those mechanisms illustrated in FIGS. 3A-3G and described in further detail below.

As illustrated in FIGS. 1-3, removable needle, or stylet, 16 is placed within the cannula lumen 38, passing through a proximal cannula port (not shown) and distal cannula port 40. Thus, cannula lumen 38 extends between both proximal cannula port (not shown) defined by a proximal cannula end 30 and a distal cannula port defined by distal cannula end 32.

Stylet 16 is slidably received in the cannula 18. It is removable and can be replaced with the same or different stylet, a composition, or another device, depending on the needs of the operator. For example, the apparatus 10 illustrated in FIG. 1 can be percutaneously inserted into the body of a subject to a desired depth and a sample of tissue cut by sharpened distal tip 28A of stylet 16. Then, stylet 16 could be removed by withdrawing it through cannula 18, leaving only cannula 18 inserted into the body of the subject. The same or different stylet 16 could be inserted back through cannula 18 for another biopsy or other use. For example, stylet 16 illustrated in FIG. 1 could be removed and replaced by a solid stylet with a blunt tip capable of conducting RF energy and, thus, cauterizing or coagulating tissue in contact with or adjacent to the stylet.

Alternatively, another device or a composition can be inserted into and through the cannula. For example, a fiber optic imager connected to a video camera can be inserted into and passed through the cannula lumen to provide images of the tissue surrounding the distal cannula end. As another example, a solid or fluid composition, such as a fluid pharmaceutical or nucleic acid composition, can be poured through the cannula to contact the tissue adjacent the distal end of the cannula.

Stylet 16 can be any suitable length and diameter; in some embodiments, stylet 16 is from about 5 cm to about 50 cm long and has a diameter of about 6 to 18 gauge.

Plural stylets also can be utilized. As one non-limiting example, instead of a single stylet, two (or more) stylets extending slidably through the cannula can be utilized. The stylets can be of the same or different in terms of their characteristics (for example, length, diameter, material construction) and can be separate or conjoined.

Cannula 18 has a proximal end 30 and a distal end 32, and can be considered a hollow needle or sleeve capable of substantially enclosing a major portion of the length of stylet 16. A grip 34 is mounted on distal end 30 of cannula 18 to assist in operation of the apparatus 10. The grip 26 on stylet 16, or the grip 34 on cannula 18, can be of different shapes and sizes than those illustrated, depending on the intended uses of the apparatus or desires of the operator.

Cannula 18 can be made from any material suitable for percutaneous insertion into the body of a subject, including (but not limited to) surgical steel, polymer, or composite materials. The cannula 18 can be made from conductive or nonconductive materials. If the cannula 18 is made from nonconductive material, a conductive material capable of transmitting RF energy is placed on the outer surface of cannula 18 at the exposed portion of distal end 32 of cannula 18 and electrically connected to an energy generator, such as an RF generator. For example (and without limitation), the cannula could be made from stiff plastic with an exposed metal foil band wrapped around its distal end and electrically connected to an RF generator through wires placed under or extending through the insulating layer 20.

Also at proximal end 30 of cannula 18, an RF conduit 36 is shown emerging from underneath insulation layer 20. One end of this RF conduit 36 is electrically coupled to cannula 18, while the other end of RF conduit 36 is electrically coupled to an RF generator (not shown). RF conduit 36 can be any type of material suitable for conducting RF energy from an RF generator to cannula 18; in some embodiments, RF conduit is an insulated wire.

RF energy, or other type of energy, can be supplied to the apparatus in any suitable manner. For example (and without limitation), a commercially available RF generator can be used to supply RF energy to the apparatus, such as the RF 3000™ Radio frequency Generator and other generators available from the RadioTherapeutics Corporation (Sunnyvale, Calif.), the Model 500 Generator and other generators available from Rita Medical Systems, Inc. (Mountain View, Calif.), or the Force™ 1C and other generators available from Valleylab, a division of Tyco Healthcare Group, LP (Boulder, Colo.). If necessary, an adapter can be used to connect the apparatus to these or other proprietary generators or other energy sources. The RF energy supplied to cannula 18 can be controlled by such an RF generator; in some embodiments, RF energy of about 480 kilohertz, from about 10 to about 200 watts, sine wave, is supplied to cannula 18 for cauterization or coagulation of tissue.

Cannula 18 can be any suitable length and diameter, though it should be sized to receive at least a major portion of the length of stylet 16. In some embodiments, cannula 18 is from about 5 cm to about 50 cm long and has an outer diameter of about 4 to 18 gauge and an inner lumen diameter of about 2 to 16 gauge.

The outer surface of cannula 18 is substantially enclosed by insulating layer 20, leaving only the distal end portion 32 exposed. Insulating layer 20 is a substantially non-conductive material that provides electrical and/or thermal insulation, such as plastic, rubber, or other polymer. Thus, when RF energy is applied to the electrically conductive cannula 18, insulation layer 20 shields the surrounding tissue from being cauterized or coagulated and allows the operator to grip the protected portion of cannula 18 with little risk of injury. The insulation layer can be any appropriate thickness, though in some embodiments, the insulating layer is thin enough to provide a low-profile, such that the gauge of the needle shaft will not be altered significantly along the insulated portion. For example (and without limitation), the insulating layer can be just thick enough such that the guage of a normally 17 guage cannula is increased to 17.5 gauge.

The exposed distal end 32 of cannula 18 is not electrically insulated, however. Thus, tissue immediately adjacent to or in contact with this exposed portion can be affected by the RF energy conducted, such as being cauterized or coagulated. Optionally, insulation layer 20 can be coated with a friction reducing material, such as silicone, to facilitate insertion of the apparatus into a subject.

The size and area of this exposed portion of distal end portion 32 can be altered for different embodiments and provides means for cauterizing tissue. In some embodiments, substantially all of the proximal half of cannula 18 is insulated by the insulation layer 20, grip 34, or both, and a major portion of the distal half of cannula 18 is covered by insulation layer 20. In the illustrated embodiment, about 2 cm of the distal end 32 of cannula 18 is free of insulation layer 20 and is exposed, though in alternative embodiments, a differently sized portion of the distal end 32 can be non-insulated, such as about 1 mm to about 5 cm of the distal end 32. In other alternative embodiments, the exposed portion is located adjacent distal end 32, while the distal tip itself is covered by insulation layer 20. In yet other alternative embodiments, only a portion of the circumference of cannula 18 is exposed at its distal end 32, rather than the entire circumference. In still other alternative embodiments, a plurality of portions of cannula 18 are exposed, such as a plurality of rings or bands of exposed cannula 18, or a plurality of individual patches of exposed cannula 18.

FIGS. 3A-3G are longitudinal cut-away views through the distal end 14 of the apparatus 10 illustrating different embodiments. In these illustrations, stylet 16 can more clearly be seen placed within the cannula lumen defined by cannula interior wall 50. The different distal tips 28A-E provide means for penetrating and/or taking biopsies of tissue.

FIG. 3A is an enlarged close-up, longitudinal cut-away view of the embodiment illustrated in FIGS. 1 and 2. FIG. 3B illustrates an embodiment similar to that in FIG. 3A, except that distal end 24B of stylet 16 includes a notch 52 for capturing additional tissue during a biopsy procedure. In FIG. 3C, distal tip 28B forms a different sharpened point than that illustrated by 28A. FIG. 3D illustrates a stylet 16 having a blunt, solid tip 28C. RF current into the stylet to heat it and melt the plug. Thus, tissue at a desired depth or location can be selectively biopsied. For example, an operator using the embodiment illustrated in FIG. 3E could percutaneously insert stylet 16 into the liver of a subject, cause the plug at distal tip 28D of stylet 16 to melt, and then take a core biopsy of the subject's liver. Thus, selective biopsy of the liver could be accomplished without coring tissue from the skin or muscles penetrated during percutaneous insertion of the apparatus.

A temperature sensor optionally can be utilized with the apparatus 10. In such an embodiment, a thermistor, thermocouple, or other temperature sensor (not shown) is placed adjacent the distal end 14 of apparatus 10, such as a thermocouple on a separate wire percutaneously inserted alongside the apparatus 10. In particular embodiments, a thermistor is mounted on the outer surface of cannula 18 adjacent its distal end 32.

A cauterization temperature of at least a minimum of 70° C. provides near-instant coagulation necrosis of the tissue in contact with the needle at this temperature. An apparatus having a temperature sensor capable of monitoring tissue temperature lining the biopsy track or adjacent to it would provide a benefit of allowing more accurate monitoring of tissue temperature during cauterization. More accurate biopsy track thermometry can decrease the risk of over-cauterization of normal tissue and can make hemostasis more reproducible.

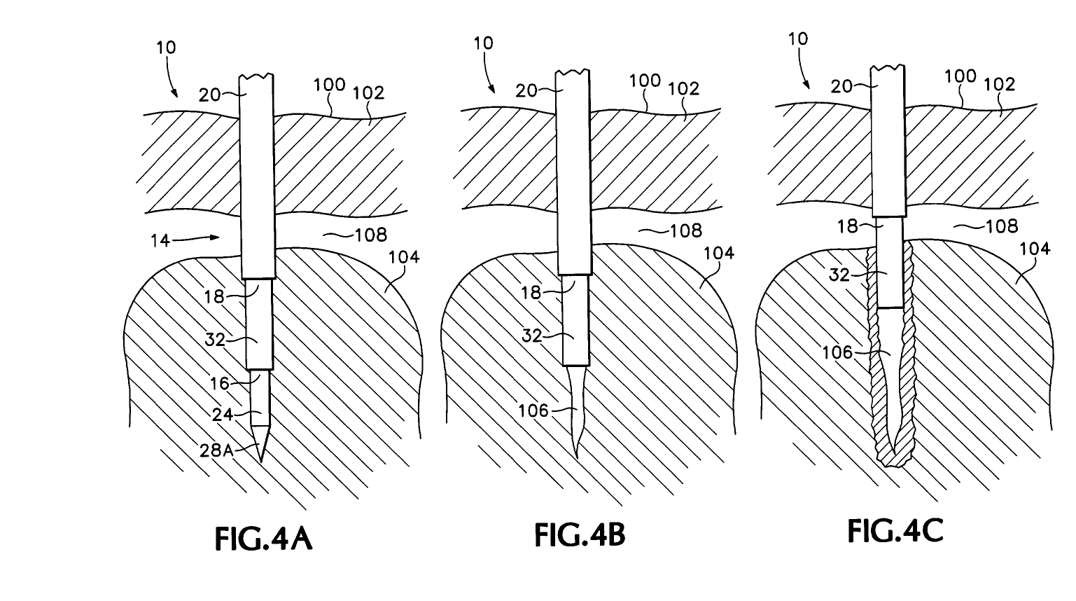

The apparatus described above can be used in a variety of percutaneous surgical applications, such as taking a biopsy or aspirating tissue. One such method is illustrated in FIGS. 4A-C. In FIG. 4A, the distal end of apparatus 10 is shown inserted into the body of a subject percutaneously through the outer body surface 100 of the subject and the surface tissues 102 through interstitial space 108 into an internal organ 104, such as a liver, kidney, lung, or other organ. In alternative embodiments, the apparatus is inserted into a neoplasm, such as a tumor, rather than an organ of the subject.

This percutaneous insertion creates a biopsy track 106, the end of which is shown in FIG. 4B (an illustration of the apparatus 10 with stylet 16 removed). After

FIG. 3E illustrates a apparatus 10 having a hollow stylet 28D. Such a stylet is useful for coring tissue during biopsy. A sharpened edge 54 is capable of cutting around and through tissue at the end of stylet 16, thus allowing a plug of tissue to enter the lumen 55 of stylet 16 via biopsy port 56.

FIG. 3F illustrates a stylet 16 having a set of biopsy forceps 28E at its distal end. Such an embodiment would be useful for cutting and removing tissue during a biopsy procedure.

FIG. 3G illustrates an embodiment similar to that illustrated in FIGS. 1-2 and 3A. However, in this embodiment, distal end 32 of cannula 18 has been beveled at 58 to facilitate insertion of the apparatus into the body of a subject.

In some embodiments, stylet 16 is electrically coupled to the RF generator to provide added length to the cauterization electrode 32 for cauterizing tissue deeper in the subject's body. Such electrical coupling could be accomplished by using a stylet of electrically conductive material, such as surgical steel, in contact with cannula 18, which is electrically coupled to the RF generator by RF conduit 36. Alternatively, the stylet could be coupled to the RF generator by a separate RF conduit, such as a wire. Thus, such a stylet provides a radio frequency probe and means for cauterizing tissue in addition to the means for cauterizing tissue associated with the cannula.

Use of the apparatus can be aided by imaging enhancement, such as a fluoroscope, ultrasound, or magnetic resonance imaging (MRI) system, to visualize internal portions of the subject's body. Thus, imaging enhancers can be placed on the apparatus to assist the operator in visualizing the apparatus via the imager. For example (and without limitation), stylet 16 can include a radiopaque marker adjacent its distal end, such as a platinum or tantalum band around its circumference, or stylet 16 can contain a ridge or channel to enhance ultrasound imaging.

In some embodiments, distal tip 28A-E of stylet 16 is protected by a plug or cap of biocompatible material, such as a collagen gel, during insertion of the apparatus into the body of the subject. The plug can be melted after insertion, for example, by passing insertion of the apparatus (as shown in FIG. 4A), stylet 16 is withdrawn (as shown in FIG. 4B). After removing stylet 16, along with a biopsy sample of tissue from organ 14, RF energy sufficient for cauterization or coagulation of tissue is supplied to cannula 18. Cannula 18 is inserted deeper into the subject, in order to cauterize or coagulate tissue surrounding the entire biopsy track, and withdrawn, as shown in FIG. 4C. Cauterized or coagulated tissue 110 adjacent cannula 18 is shown in FIG. 4C as the area surrounding biopsy track 106. RF energy is supplied while cannula 18 is withdrawn entirely, thus cauterizing or coagulating tissue along the entire biopsy track. Thus, when cannula 18 is completely withdrawn, cauterized or coagulated tissue 110 would appear along biopsy track 106 through the surface tissues 102 of the subject. Cauterizing or coagulating the biopsy track can reduce the amount of blood loss suffered by the subject (as described in further detail in the Example below), and/or reduce the likelihood of metastasizing or seeding of a malignant tumor along the biopsy track.

The temperature used for cauterization or coagulation of the biopsy track can be controlled by adjusting the RF energy supplied to the cannula by the RF generator, adjusted according to the needs and desires of the operator, and optionally measured via a thermocouple or thermistor. In some embodiments, the cauterization/coagulation temperature is about 100° C. or less, such as about 80° C. or less, such as about 70° C. In particular embodiments, the cauterization/coagulation temperature is from about 70° C. to about 100° C.

### EXAMPLE

Cauterization of the needle track following coaxial needle biopsy can decrease hemorrhage following ultrasound-guided liver biopsy.

Methods and Materials

A 14 gauge 10 cm coaxial introducer needle with a removable pencil-point stylet was modified to serve as a radio frequency ablation electrode following core biopsy. The proximal 8 cm of the metal shaft was electrically insulated by non-conducting heat-shrinkable plastic extending around the circumference. Electrical connection with the needle was established by encasing the bare end of an 18 ga wire within the plastic sheath in direct contact with the needle. The distal 2 cm of the shaft at the tip remained exposed to act as a monopolar electrode for deposition of alternating current in the radio frequency range. The outer diameter of the insulated portion of the needle was 3.0 mm or approximately 11 gauge.

The needle was tested in five crossbred domestic swine (weight range: 177-260 pounds) under an animal use protocol approved by the Institutional Animal Care and Use Committee of the Food and Drug Administration Centery for Veterinary Medicine. General anesthesia was induced with a mixture of telazol, xylazine ketamine, atropine and torbugesic and maintained with 1-3% isoflurane, following endotracheal intubation. The pigs were placed in a left lateral decubitus position and a combined laparotomy and thoracotomy was performed to expose the liver. The right kidney was also exposed in two pigs. Four disposable single foil ground pads were placed on each pig, one on each shoulder and hip, and both the grounding pads and the modified introducer needle were connected to a 200 watt, 480 kilohertz radio frequency generator (CC 1 Cosman coagulator system, Radionics, Burlington, Mass.).

Multiple biopsies were performed on each animal in the study. For each biopsy, the modified introducer needle was inserted into the liver without imaging guidance. A 22 gauge spinal needle was placed adjacent to the shaft of the introducer needle with the tip of the spinal needle within 1 cm of the tip of the introducer needle. The position of the introducer needle was confirmed by ultrasound and a thermistor (TCA-2, Radionics, Burlington, Mass.) was inserted through the spinal needle. A biopsy was performed through the introducer needle with a standard unmodified 16 gauge biopsy gun with a 20 mm notch (Temno, Allegiance) deployed and fired through the modified introducer needle. Randomization to no radio frequency ablation (No RF group) or radio frequency ablation (RF group) occurred following biopsy. In the No RF group, both needles were simultaneously withdrawn. In the RF group, the RF current was applied to the outer needle as the needle was slowly withdrawn from the liver, with a goal of maintaining the temperature between 70 and 100° C. In another animal, the withdrawal of the introducer needle was paused for 15-30 seconds for longer ablation of the distal 2 cm of the needle track. In the RF group in this animal, the temperature did exceed 100° C. in two samples. For both the No RF and the RF groups, the blood loss at the liver surface was measured by collecting the blood in pre-weighed gauze pads for exactly two minutes and recording the differences in measured weight.

In two animals, the modified introducer needle was inserted into the kidney perpendicular to the surface and in the plane of symmetry of the kidney without imaging guidance. A 22 gauge spinal needle was placed adjacent to the shaft of the introducer needle with the tip of the spinal needle within 1 cm of the tip of the introducer needle. The position of the needle was confirmed by ultrasound and a thermistor was inserted through the spinal needle. A biopsy was performed through the introducer needle with the 16 gauge biopsy gun deployed and fired through the modified introducer needle. Randomization to no radio frequency ablation (No RF group) or radio frequency ablation (RF group) occurred following biopsy. In the No RF group, both needles were simultaneously withdrawn. In the RF group, the RF current was applied to the outer needle as the needle was slowly withdrawn from the kidney, with a goal of maintaining the temperature between 70 and 100° C. For both the No RF and the RF groups, the blood loss at the kidney surface was measured by collecting the blood in pre-weighed gauze pads for exactly two minutes and recording the differences in measured weight.

Statistical Evaluation.

At least 7 and no more than 12 liver biopsies were performed on each animal in the study. The treatments (RF or No RF) were randomly assigned within each animal for each biopsy site. Randomization and treatment assignment were blinded until after each biopsy was performed. For each animal, the difference between the average weight of the blood collected at the biopsy needle exit wound sites with and without RF ablation was calculated. This difference was used in the statistical analysis to remove the between animal variation (for example, blood clotting time, blood pressure). A two-tailed Student's t-test was used to determine if the difference between the treatments was significantly different from zero. One data point that was more than 5 standard deviations greater than the mean of all the individual weights in that animal's No RF treatment group was removed as an outlier. This data point would have numerically increased the difference between the two treatment groups, but it also would have increased the treatment variation 3-fold. Results are presented as means±SEM. Probability values of <0.05 were considered statistically significant.

For the renal ablations, a two-tailed Student's t-test was used to determine if the difference between the treatments was significantly different from zero. Results are presented as means±SEM. Probability values of <0.05 were considered statistically significant.

Results

Ablation using radio frequency energy during withdrawal of the introducer needle resulted in thermal coagulation of the needle track as compared to simple biopsy without RF ablation.

Mean blood loss, number of biopsy sites, and total number of biopsies for the RF and No RF groups in each animal are presented in Table 1 along with the difference between the mean blood loss in the two groups or delta. The aggregate data for weight of blood for all pigs was 6.4±1.1 gm in the No RF group and 2.3±0.7 gm in the RF group, with a delta of 4.1±0.9 gm. The biopsies in the RF group had less bleeding than those in the No RF group, p<0.03.

TABLE 1
Biopsy Needle Ablation Data

| | |
|---|---|
| Mean Blood Loss | Number of |
| for Biopsy Sites | Biopsy Sites |
| (grams of blood) | per Animal |

| Animal | no RF | RF | Delta | No RF | RF | Total |
|---|---|---|---|---|---|---|
| 1 | 7.9 | 0.9 | 7.0 | 3 | 4 | 7 |
| 2 | 3.2 | 2.0 | 1.3 | 6 | 6 | 12 |
| 3 | 4.5 | 1.1 | 3.4 | 5 | 4 | 9 |
| 4 | 8.8 | 4.6 | 4.3 | 5 | 5 | 10 |
| 5 | 7.5 | 2.8 | 4.7 | 4 | 6 | 10 |
| Mean | 6.38 | 2.26 | 4.12 | 4.60 | 5.00 | 9.60 |
| SEM | 1.08 | 0.68 | 0.93 | 0.51 | 0.45 | 0.81 |

Having illustrated and described the principals of the invention by several embodiments, it should be apparent that those embodiments can be modified in arrangement and detail without departing from the principles of the invention. For example, RF energy could be conducted to the distal end of a biopsy apparatus by a conductive path that is associated with a cannula, but that is other than the entirety of the cannula. Thus, the invention includes all such embodiments and variations thereof, and their equivalents.

1. A biopsy apparatus, comprising:
a hollow cannula having a proximal cannula end and a distal cannula end, the cannula defining a lumen, a first port adjacent the proximal end of the hollow cannula and a second port adjacent the distal end of the hollow cannula, with the cannula lumen extending between the ports, wherein the cannula has a conductive pathway capable of transmitting cauterization energy between the proximal end and the distal end with at least a portion of the conductive pathway being electrically insulated from the surrounding environment;
a thermistor operably coupled to the distal cannula end;
an elongated stylet sized for insertion into the cannula lumen; and
a source of cauterization energy operably coupled to the conductive pathway, the source being adjustable in response to the thermistor so that the amount of cauterization energy applied to the distal end can be controlled.

2. The biopsy apparatus according to claim 1 wherein the first port is located at the proximal end of the cannula and the second port is located adjacent the distal end of the hollow needle.

3. The biopsy apparatus according to claim 1 wherein:
the cannula has an outer surface that is at least partially surrounded by a layer of electrically insulating material; and
the insulating material insulates a major portion of the distal half of the outer surface of the cannula.

4. The biopsy apparatus according to claim 1 wherein the elongated stylet further comprises a sharpened tip or biopsy forceps.

5. The biopsy apparatus according to claim 1 wherein the cannula or the stylet further comprises an image enhancer.

6. The biopsy apparatus according to claim 1 wherein the stylet is slidably received within the cannula lumen.

7. The biopsy apparatus according to claim 1 wherein the cauterization energy is radio frequency energy.

8. The biopsy apparatus according to claim 1, further comprising a handgrip coupled to the proximal portion of the cannula.

9. An apparatus for cauterizing a needle track produced during core biopsy, comprising:
a needle;
a hollow sleeve having a portion capable of transmitting cauterization energy comprising a proximal end and a distal end, a first port defined by the proximal end and a second port defined by the distal end, and a sleeve lumen sized to receive the needle, wherein the sleeve lumen extends between the first and second ports;
an insulator substantially enclosing a portion of the hollow sleeve; and
a source of cauterization energy operably coupled to the hollow sleeve.

10. The apparatus according to claim 9 wherein the needle is a solid needle.

11. The apparatus according to claim 9 wherein the needle further comprises a sharpened tip or biopsy forceps.

12. The apparatus according to claim 9 wherein the insulator encloses a major portion of the distal half of the hollow sleeve.

13. The apparatus according to claim 9 wherein the cauterization energy is radio frequency energy.

14. A biopsy apparatus, comprising:
an inner needle comprising means for penetrating tissue;
a hollow outer needle comprising means for cauterizing tissue and further comprising a proximal outer needle end, a distal outer needle end, and an outer needle lumen, wherein the lumen extends between a first port adjacent the proximal end of the hollow needle and a second port adjacent the distal end of the hollow needle, and wherein a portion of the outer needle between the proximal end and distal end comprises an insulating material; and
a source of cauterization energy operably coupled to the outer needle

15. The biopsy apparatus according to claim 14 wherein the means for cauterizing tissue is a radio frequency energy probe.

16. The biopsy apparatus according to claim 14 wherein the inner needle further comprises means for biopsying tissue.

17. The biopsy apparatus according to claim 16 wherein the means for biopsying tissue comprises a sharpened tip, sharpened edge, or biopsy forceps.

18. The biopsy apparatus according to claim 14 wherein the cauterization energy is radio frequency energy.

19. A method for taking a tissue biopsy, comprising:
inserting the distal end of the apparatus according to claim 1 into the tissue of a subject, thereby forming a biopsy track;
removing a tissue biopsy from the subject; and
withdrawing the distal end of the apparatus according to claim 1 from the subject while supplying cauterization energy to the apparatus, wherein the cauterization temperature is less than 100° C.

20. The method according to claim 19 wherein the subject is a mammal.

21. The method according to claim 19 wherein the subject is a human.

22. The method according to claim 19 wherein the tissue comprises a neoplasm or the liver of the subject.

23. The method according to claim 19 wherein the cauterization energy is radio frequency energy.

24. The method according to claim 19 wherein the temperature is greater than or equal to about 70° C.

25. The method according to claim 24 wherein the temperature is from about 70 to about 80° C.

26. The method according to claim 25 wherein the temperature is about 70° C.
